(19) United States
(12) Patent Application Publication
(10) Pub. No.: US 2024/0084355 A1
(43) Pub. Date: Mar. 14, 2024

(54) CELL CULTURE METHODS

(71) Applicant: DR. REDDY’S LABORATORIES LIMITED, Hyderabad (IN)

(72) Inventors: Rama Bhupal Reddy KANDULA, Hyderabad (IN); Suman BANDYOPADHYAY, Secunderabad (IN); Vikas CHANDRAWANSHI, Hyderabad (IN); Sampath kumar VEERAMALLY, Hyderabad (IN); Raghavendra POLISHETTY, Roudkunda Sindhanur Raichur District (IN); Megha BS, Hyderabad (IN)

(21) Appl. No.: 18/275,255

(22) PCT Filed: Jan. 30, 2022
(86) PCT No.: PCT/IN2022/050078

§ 371 Date: Aug. 1, 2023

(30) Foreign Application Priority Data
Feb. 1, 2021 (IN) 202141004385

(51) Int. Cl. C12P 21/00; C07K 14/705

(52) U.S. Cl. CPC C12P 21/005; C07K 14/70521; C07K 2319/30

(57) ABSTRACT

The present invention discloses a method of cell culture for producing a fusion glycoprotein composition comprising a target glycosylation profile. More particularly, the invention provides a process to produce a glycoprotein composition from mammalian cell culture, wherein the composition comprises a target total sialylated and or di- and tri-sialylated N-glycan variant.

### FIELD OF INVENTION

The Biological material used in the invention was not obtained from India. The present invention relates to cell culture methods. In particular the cell culture process relates to production of Fc-fusion protein compositions characteristed by particular levels of sialylated glycoforms.

### BACKGROUND OF THE INVENTION

Therapeutic glycoproteins form, one of the mainstays in the broader class of biotherapeutics, that have been approved for treatment of various human disorders. Although these glycoproteins can be produced in non-mammalian expression systems (e.g bacterial, yeast, plant, insect expression systems), mammalian expression systems are the preferred production platforms. One of the main factors that weighs in their favour is that mammalian expression systems can produce glycoproteins that have post-translational modifications (PTMs) that are similar to humans.

PTMs refer to enzymatic modification of proteins following its translation which generally result in mature protein. The PTMs can include addition of chemical moeities to target proteins which range from addition of simple smaller groups as in phosphorylation, methylation, acetylation, hydroxylation, to addition of complex biomolecules as in glycosylation, prenylation etc. or addition of polypeptide as in ubiquitination. The PTMs can result in modification of amino acid residues in protein and also in proteolytic degradation of protein. As a result of these modifications, the structure, stability and function of the proteins are altered.

Amongst various PTMs in therapeutic glycoproteins, glycosylation has been found to have significant effect on properties relevant to their therapeutic application. Glycosylation in therapeutic glycoproteins has been reported to affect their biological activity, efficacy, stability, immunogenicity, clearance rate, antibody-dependent cellular cytoxicity (ADCC), and complement-dependent cytoxicity (CDC).

The two major types of glycosylation in mammalian expression systems are N-linked glycosylation, in which glycans are attached to the asparagine of the recognition sequence Asn-X-Thr/Ser, where “X” is any amino acid except proline, and O-linked glycosylation in which glycans are attached to serine or threonine.

The structural heterogeneity in glycosylation can be either due to diversity in glycan structure on a particular glycosylation site (microheterogeneity) or due to variablilty in occupancy of the possible glycosyaltion sites in protein sequence (macroheterogeneity). This may be of consequence of the competitive action of diverse enzymes involved in glycosylation and are key to understanding glycoprotein heterogeneity (Mariño, K., (2010) Nature Chemical Biology 6, 713-723).

Given the significant impact glycosylation can have on the safety and efficacy of therapeutic glycoproteins, the regulatory agencies ascribe much importance to characterisation of these critical quality attributes. Minimising heterogeneity in therapeutic glycoprotein preparations is a continuous challenge and thus, the analytical profile of the composition, particularly the glycosylation needs to be closely monitored at all stages of drug development. In case of biosimilars, this is a heightened requirement, in the sense that, they have to address the issue of heterogeneity in batches as well as high similarity with the reference biological product.

The final glycosylation profile in a given composition is not only influenced by the in vivo factors (viz. the specific cell line of production and the intracellular glycosylation machinery) but also by in vitro factors such as cell culture conditions and the multiple levels of upstream and downstream processing that the protein is subjected to. The effect of these factors on glycosylation profile vary not only with the individual glycoprotein but also with the different glycovariants of same therapeutic glycoprotein. Hence, these factors need to be optimised so as obtain a therapeutic glycoprotein with desired glycoprofile.

CTLA4-Ig fusion protein is a highly glycosylated protein that contains multiple N- and O-glycosylation sites resulting into structurally complex glycans. Also, these glycan species are highly branched (especially the N-linked species) and terminally sialylated (in both N- and O-linked glycans). All these results in the complexity for developing CTLA-4 fusion protein with acceptable quality, efficacy and safety.

### SUMMARY OF THE INVENTION

The present invention discloses a cell culture method for producing a CTLA-4 fusion protein composition comprising target sialylated N-glycans. More particularly, the invention provides a cell culture process comprising culturing the mammalian to obtain a glycoprotein composition from mammalian cell culture, wherein the composition comprises reduced sialylation or reduced sialylated N-glycan content or reduced di- and tri-sialylated N-glycans content and/or total sialylated glycans.

### BRIEF DESCRIPTION OF DRAWINGS

FIG. 1. Schematic representation of the primary protein structure of a CTLA-4-IgG fusion protein which exists as dimer of identical monomeric polypeptide chains. Each monomeric polypeptide chain consists of 357 amino acids, of which residues 1 to 125 forms the extra cellular CTLA-4 domain, while residues 126 to 357 forms IgG HC Fc domain.

FIG. 2. Viable cell density (VCD) of the cell cultures as described in Examples I, II and III.

FIG. 3. pCO₂ levels of the cell cultures as described in Examples I, II and III.

### DESCRIPTION OF THE INVENTION

### Definitions

The term “about” refers to a range of values that are similar to the stated reference value to a range of values that fall within 25, 20, 19, 18, 17, 16, 15, 14, 13, 12, 11, 10, 9, 8, 7, 6, 5, 4, 3, 2, 1 percent or less of the stated reference value.

The terms “additive” or “supplement” as used herein refer to any supplementation made to cell culture medium/feed to achieve the goals described in this disclosure. An “additive” or “supplement” can include a single substance, e.g., galactose, insulin, glucose or fucose, or can include multiple substances, e.g., galactose, insulin, and fucose; or galactose and insulin; or galactose and fucose.

The terms “cell culture medium”, “culture medium”, “media”, “medium”, as used herein refer to a solution containing nutrients which are required to support the growth of the cells in cell culture. The term would include basal medium which is typically used to support the cell growth during the initial growth phase of cell culture. It would also include feed medium which is typically used to support the later growth phase and production phase of cell culture.

The term “cell culture process” as used herein refers to a process of culturing a population of cells that are capable of producing recombinant protein of interest.

The term “control” herein the present invention refers to the cell culture process of culturing a population of cells that are capable of producing recombinant protein of interest without the involvement of the inventive steps.

The term “fusion protein composition” refers to a population of fusion protein molecules or fragments thereof that is produced by mammalian cell culture. The population of fusion protein molecules may have one or several post translational modifications (PTM), imparting the fusion protein molecules a different molecular weight, charge, solubility or combinations thereof.

The term “glycan” refers to monosaccharide or polysaccharide moiety attached to another molecule.

The term “glycoform” or “glycovariant” used interchangeably herein refers to different molecular variants of a glycoprotein resulting due to variable glycan structure attached and/or glycan attachment site occupancy on the glycoprotein.

The term “glycoprotein” refers to any polypeptide or protein which has one or more covalently attached glycan.

The term “glycosylation profile” or “glycan profile” or “glycoform composition” as used herein refers to the different glycoforms or glycans species present in a glycoprotein composition and their quantities or percentages.

The term “growth phase” used herein refers to the phase in the cell culture, wherein cells actively proliferate and there is an exponential increase in cell density. In cell cultures expressing recombinant protein, during this phase cellular proliferation is the primary activity as compared to protein production.

The term “N-glycans” or “N-linked glycan” as used herein refers to the N-linked glycosylated glycans in which glycans are attached to the asparagine of the recognition sequence Asn-X-Thr/Ser, where “X” is any amino acid except proline.

The term “pCO₂ levels” used herein refer to the partial pressure of dissolved carbon dioxide (pCO₂) in the cell culture.

The term “post-translational modification” or “PTM”, herein used interchangeably, refers to biochemical modification that occurs at one or more amino acids on a protein molecule after translation of the protein. PTMs are mostly chemical or enzyme-mediated, at specific target sequences in the protein and comprise inter alia, glycosylation, glycation, acetylation, amidation, deamidation, methylation, ADP-ribosylation and hydroxylation.

The term “production phase” used herein refers to the phase in the cell culture wherein the cellular proliferation slows down to and in cells expressing a recombinant protein, the protein production is the primary activity as compared to cellular proliferation.

The term “sialylated N-glycan” as used herein refer to the N-glycan species which have sialic acid in terminal positions. This may include mono-, bi-, and/or tri-antennary glycans with mono, di, tri and tetra sialylated glycans. The glycoprotein composition encompassed by the present invention may have one or more sialylated N-Glycan species represented in Table 1.

The term “stringent” as used herein in relation to cell culture parameter means that the parameter has to be strictly maintained in particular range for their effect. The values can vary by up to 0.5% to 2.0%. For example stringent pH of 7.0 would include pH of 7.0±0.035 to 7.0±0.14.

The term “sugar” used herein refers to carbohydrates selected from sucrose, lactose, trehalose, galactose or glucose.

The term “supplementing” as used herein refers to any supplementation made to cell culture medium/feed to achieve the goals described in this disclosure.

The term “target glycosylation profile” as used herein refers to predetermined, characteristic glycosylation profile of glycoprotein composition in terms of individual glycoforms and/or their amounts present in that composition. For example “target sialylation profile” of a glycoprotein would refer to a predetermined, characteristic sialylation profile of the glycoprotein in terms of individual sialylated glycoforms/and or their amounts present in that glycoform composition. These target glycosylation profile can be based on existing monographs for that glycoprotein, approved specification for the glycoprotein by regulatory agencies, or a quality control criterion developed for pharmaceutical preparation of that glycoprotein. The amounts of the individual glycoform in this target glycosylation profile can be absolute numerical values or a range of numerical values.

The term “temperature shift” refers to the change in culture temperature during the cell culture process.

The “viable cell density” or “VCD” is defined as number of live cells in the total cell population.

The term “viability” or “cell viability” refers to percentage of viable cells out of total no. of cells including the viable and non-viable cells at a particular time point in cell culture.

The present invention discloses glycoprotein composition comprising CTLA-4-IgG fusion protein with a glycosylation profile of the protein.

A person of ordinary skill in the art would be able to perform the cell culture method described herein using the state of the art techniques. Unless defined otherwise, all technical and scientific terms used herein have the same meaning as commonly understood by a person having ordinary skill in the art to which the invention pertains.

The present invention provides mammalian cell culture process comprising use of cell culture medium, feed or additives and various culture parameters for producing fusion glycoprotein composition having target glycosylation profile. In particular, the invention provides a cell culture process comprising culturing mammalian cells to obtain a glycoprotein composition, wherein the composition comprises reduced sialylation or reduced sialylated N-glycan content or di- and tri-sialylated N-glycans content and/or total sialylated glycans.

Any mammalian cell or cell type which is suitable for expression of recombinant proteins in a cell culture medium may be used for the present invention. Non-limiting examples of mammalian cells that may be used with the present invention include Chinese hamster ovary (CHO) cells, baby hamster kidney (BHK21) cells and murine myeloma cells (NS0 and Sp2/0) human retinoblasts (PER.C6 cell line), human embryonic kidney cell line (HEK-293 cell line) (Dumont, J., et al., Human cell lines for biopharmaceutical manufacturing: history, status, and future perspectives. Crit Rev Biotechnol, 2016. 36(6): p. 1110-1122). In a preferred embodiment, CHO cell lines expressing recombinant proteins may be used in accordance with the present invention.

Cell culture medium is understood by those skilled in the art to refer to a nutrient solution in which cells, such as animal or mammalian cells, are grown. A cell culture medium generally includes one or more of the following components: an energy source (e.g., a carbohydrate such as glucose); amino acids; vitamins; lipids or free fatty acids; and trace elements, e.g., inorganic compounds or naturally occurring elements in the micromolar range. Cell culture medium can also contain additional components, such as hormones and other growth factors (e.g., insulin, transferrin, epidermal growth factor, serum, and the like); salts (e.g., calcium, magnesium and phosphate); sugars (e.g. mannose, galactose, fucose); amino acids (glutamine); buffers (e.g., HEPES); nucleosides and bases (e.g., adenosine, thymidine, hypoxanthine); antibiotics (e.g., gentamycin); and cell protective agents (e.g., a Pluronic polyol (Pluronic F68). Commercially available media can be utilized in accordance with the present invention, for example, Dulbecco's Modified Eagles Medium (DMEM, Sigma-Aldrich); RPMI-1640 Medium (Sigma-Aldrich); EX-CELL® Advanced CHO Fed-batch Medium (Sigma-Aldrich); Cell Boost™ 7a and 7b (GE Healthcare Bio-Sciences AB). One skilled in the art would appreciate that some cell culture media are suited to support cells through their initial growth phase (basal medium) while some sustain cells through the later growth phase and production phase of cell culture (feed medium), and would be able to choose appropriate culture medium.

The methods described in the present invention are in recognition of the fact that various parameters of the cell culture process may be used to obtain fusion protein composition of desired glycosylation profile.

A person of ordinary skill in the art would be able to determine the glycosylation profile of the fusion protein composition produced by the cell culture process described herein using the state of the art techniques (Ruhaak, L. R., et al., Glycan labeling strategies and their use in identification and quantification. Anal Bioanal Chem, 2010. 397(8): p. 3457-81; Wuhrer, M., A. M Deelder, and C. H. Hokke, Protein glycosylation analysis by liquid chromatography-mass spectrometry. J Chromatogr B Analyt Technol Biomed Life Sci, 2005. 825(2): p. 124-33; Guile, G. R., et al., A rapid high-resolution high-performance liquid chromatographic method for separating glycan mixtures and analyzing oligosaccharide profiles. Anal Biochem, 1996. 240(2): p. 210-26).

In an embodiment, the invention discloses a cell culture method for the production of a fusion protein composition, wherein one or more cell culture parameters selected from cell culture media, cell culture additives, temperature, pH, pCO₂ are optimised, so as to obtain the fusion protein composition having a target glycoprofile. In an embodiment, the target glycoprofile includes glycovariants selected from the list comprising afucosylated variant, high mannose variant, galactosylated variant, and sialylated variant. In a preferred embodiment, the target glycoprofile includes glycovariants selected from the list comprising high mannose variant, galactosylated variant, and sialylated glycans.

In another embodiment, the fusion protein composition so obtained from the cell culture method of the present invention is composed of target levels of total sialylated glycovariants. In a preferred embodiment, the fusion protein composition so obtained from the cell culture method is composed of target levels of total sialylated N-glycan and/or di+tri sialylated N-glycan variants.

In an embodiment, the cell culture process of present invention would comprise use of a temperature shift so as to obtain fusion protein composition with the target glycoprofile. In an embodiment, the cell culture process of the present invention would comprise more than one temperature shift, wherein the individual temperature shift might be result in subsequent lower temperature or higher temperature. For example in a cell culture process having two temperature shift, the following combinations are encompassed: 1^{st} high temperature→2^{nd} low temperature 43^{rd} lower temperature, 1^{st} high temperature→2^{nd} low temperature→3^{rd} high temperature, 1^{st} low temperature→2^{nd} high temperature→3^{rd} low temperature. In a preferred embodiment, the cell culture method of present invention includes a single temperature shift, wherein the second temperature is lower than the first temperature. In yet another embodiment, the cell culture method of present invention includes a single temperature shift which marks the start of production phase of cell culture.

In an embodiment, the cell culture method of present invention includes maintaining stringent pH during the cell culture so that the fusion protein composition so obtained has the target glycoprofile. In yet another embodiment, the invention discloses a cell culture method for production of fusion protein composition with target glycoforms wherein the process includes maintaining the pCO₂ within defined ranges. In a preferred embodiment, the cell culture method of the present invention includes modulation of pCO₂ levels in two different ranges during the growth phase and production phase of the cell culture.

In an embodiment, the invention discloses a cell culture method for the production of a fusion protein composition comprising target sialylated N-glycans, the said method comprising
a) providing/culturing mammalian cells expressing the said fusion protein,
b) culturing the cells at a stringent pH value,
c) maintaining higher pCO₂ levels during the production phase than the pCO₂ levels during the growth phase
d) subjecting the cell culture to at least one temperature shift,
e) recovering the recombinant fusion protein from the culture.

In another embodiments, the invention discloses a cell culture method for the production of a fusion protein composition comprising sialylated N-glycans, the said method comprising
a) providing/culturing mammalian cells expressing the said fusion protein,
b) culturing the cells at a stringent pH value,
c) maintaining higher pCO₂ levels during the production phase than the pCO₂ levels during the growth phase
d) culturing the cells at first temperature for a first period of time, subjecting the cell culture to a temperature shift, wherein the second temperature is lower than the first temperature
e) supplementing the cell culture medium with at least one sugar
f) recovering the said fusion protein composition from the culture,
   wherein, the sialylated N-glycan content of the fusion protein composition is reduced as compared to sialylated N-glycan content of the fusion protein composition produced by a similar method devoid of step b), step c) and step d).

In another embodiments, the invention discloses a cell culture method for the production of a fusion protein composition having a target sialylated N-glycan content, the said method comprising
a) providing/culturing mammalian cells expressing the said fusion protein,
b) culturing the cells at a stringent pH value,
c) maintaining higher pCO₂ levels during the production phase than the pCO₂ levels during the growth phase
d) culturing the cells at first temperature for a first period of time, subjecting the cell culture to a temperature shift, wherein the second temperature is lower than the first temperature
e) supplementing the cell culture medium with at least one sugar
f) recovering the said fusion protein composition from the culture,
   wherein, the target sialylated N-glycan content of the fusion protein composition is about 21% to 38% lower than the sialylated N-glycan content of the fusion protein composition produced by a similar method devoid of step b), step c) and step d).

In another embodiments, the invention discloses a cell culture method for the production of a fusion protein composition comprising sialylated N-glycans, the said method comprising
a) providing/culturing mammalian cells expressing the said fusion protein,
b) culturing the cells at a pH of 7.0±0.1,
c) maintaining higher pCO₂ levels during the production phase than the pCO₂ levels during the growth phase
d) culturing the cells at first temperature for a first period of time, subjecting the cell culture to a temperature shift, wherein the second temperature is lower than the first temperature
e) supplementing the cell culture medium with at least one sugar
f) recovering the said fusion protein composition from the culture,
   wherein, the sialylated N-glycan content of the fusion protein composition is reduced as compared to sialylated N-glycan content of the fusion protein composition produced by a similar method devoid of step b), step c) and step d).

In another embodiments, the invention discloses a cell culture method for the production of a fusion protein composition comprising sialylated N-glycans, the said method comprising
a) providing/culturing mammalian cells expressing the said fusion protein,
b) culturing the cells at a pH of 7.0±0.1,
c) maintaining the growth phase pCO2 levels at about 20 mmHg to about 50 mmHg and increasing the pCO2 levels to 100 to 135 mmHg during the production phase
d) subjecting the cell culture to a temperature shift from about 37° C. to 34° C. on day 6 of cell culture
e) supplementing the cell culture medium with at least one sugar
f) recovering the said fusion protein composition from the culture,
   wherein, the sialylated N-glycan content of the fusion protein composition is reduced as compared to sialylated N-glycan content of the fusion protein composition produced by a similar method devoid of step b), step c) and step d).

In another embodiments, the invention discloses a cell culture method for the production of a fusion protein composition comprising sialylated N-glycans, the said method comprising
a) providing/culturing mammalian cells expressing the said fusion protein,
b) culturing the cells at a pH of 7.0±0.1,
c) maintaining the growth phase pCO2 levels at about 20 mmHg to about 50 mmHg and increasing the pCO2 levels to 100 to 135 mmHg during the production phase
d) subjecting the cell culture to a temperature shift from about 37° C. to 34° C. on day 6 of cell culture
e) supplementing the cell culture medium with at least one sugar
f) recovering the said fusion protein composition from the culture,
   whereby the resultant fusion protein composition comprises about 20.83% di- and tri-sialylated N-glycans content and/or about 50.82% total sialylated glycans.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

TABLE 1
Representative Sialylated N-Glycan species.

| Species name | Formula |
|---|---|
| Di + tri | G2FS2, G2FS2(NGNA1), G2FS2-Ac1A, G2FS2-Ac1B, |
| Sialylated | G2FS2-Ac2A, G2FS2-Ac2B, Tetra-Ant-G4FS2, Tetra- |
| | Ant-G4FS3A, Tetra-Ant-G4FS3B, Tri-Ant-G3FS2, Tri- |
| | Ant-G3FS2(NGNA1), Tri-Ant-G3FS3A, Tri-Ant-G3FS3- |
| | Ac1, Tri-Ant-G3FS3B, [Tri-Ant-G2FS2A + Tri-Ant- |
| | G2FS2(-NAG)] |
| Total Sialylated | G1FS1A, G1FS1B, G2FS1A, G2FS1-Acl, G2FS1Ac2, |
| | G2FS1B, G2FS2, G2FS2(NGNA1), G2FS2-Ac1A, |
| | G2FS2-Ac1B, G2FS2-Ac2A, G2FS2-Ac2B, Tetra-Ant- |
| | G4FS2, Tetra-Ant-G4FS3A, Tetra-Ant-G4FS3B, Tetra- |
| | Ant-G4FS4, TriAnt-G2FS1(-NAG), Tri-Ant-G3FSlA, |
| | Tri-Ant-G3FS2, Tri-Ant-G3FS2(NGNA1), Tri-Ant- |
| | G3FS3A, Tri-Ant-G3FS3-Ac1, Tri-Ant-G3FS3B, [Tri- |
| | Ant-G2FS2A + Tri-Ant-G2FS2(-NAG)], G2FS1(NGNA) |

### EXAMPLES

### Example I

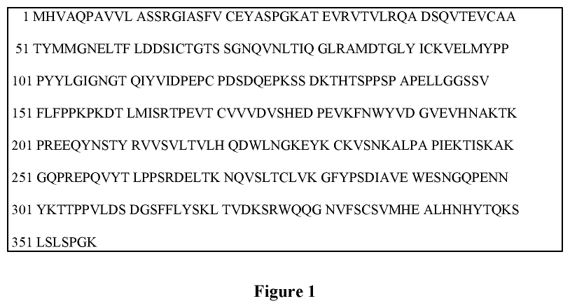

A CTLA-4-IgG fusion protein, having a primary protein structure as depicted in FIG. 1, was cloned and expressed in a CHO cell line techniques described in detail in “Molecular Cloning: A Laboratory Manual (Fourth Edition)”. The cell culture was initiated by seeding the cells at seeding density of 0.5 million cells/ml in EX-CELL® Advanced CHO Fed-Batch Medium (Catalogue no. 24366C, Sigma-Aldrich, USA). The cell culture medium was supplemented with glucose, which is made up to a concentration of 5 g/L every day. A total of 18% (v/v) feed medium (BalanCD CHO Feed 4 (Catalogue no. 94134, Irvine Scientific, USA)) was added on day 3 (3%), 5 (5%), 7 (4%), 9 (3%), 11 (1.5%) and 12 (1.5%). The cell culture was initiated at temperature 37° C. and a dual temperature shifts were performed. The first shift from 37° C. to 34° C. was done on day 5 and second shift from 34° C. to 30° C. was performed on day 9. The cell culture was maintained at a relaxed pH of the 7.0±0.40 using 1 M sodium carbonate and C02 sparging. The culture was harvested on day 14 or at cell viability greater than 80%. The experiment was run in 4 different batches.

### Example II

The cell culture process described in Example I was carried with following modifications. The cell culture was initiated at temperature 37° C. and a single temperature shift was performed from 37° C. to 34° C. on day 6. In addition, the cell culture was maintained at a relaxed pH of the 7.15±0.25. The culture was harvested on day 14 or 15 or at cell viability greater than 80%. The experiment was run in 2 different batches.

### Example III

The cell culture process described in Example I was carried with following modifications. The cell culture was initiated at temperature 37° C. and a single temperature shift was performed from 37° C. to 34° C. on day 6. Further, the cell culture was maintained at a stringent pH of the 7.0±0.1. The culture was harvested on day 14 or 15 or at cell viability greater than 80%. The experiment was run in 4 different batches.

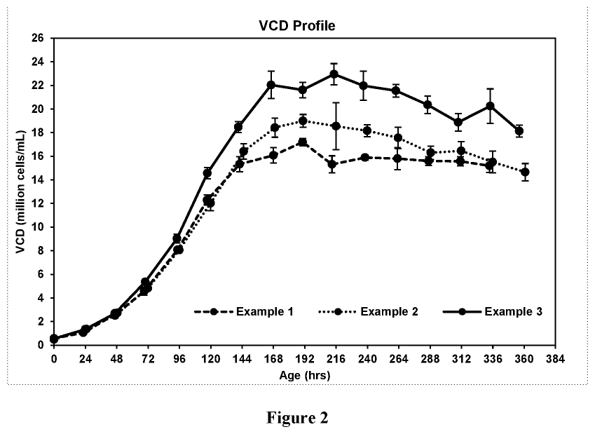

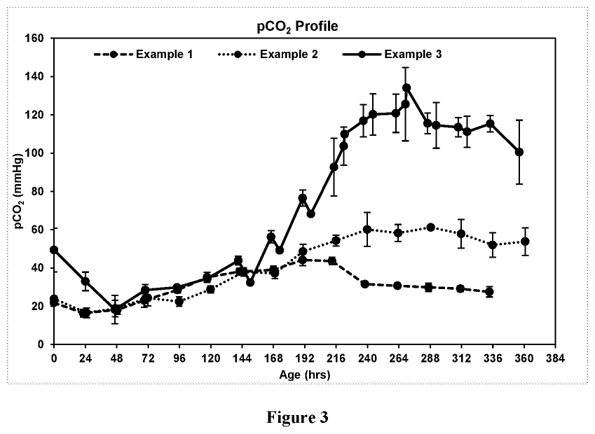

The viable cell density (VCD) and the dissolved pCO₂ levels of the cell culture described in Example I, Example II, and Example III is represented in FIG. 2 and FIG. 3 respectively. Table 2 provides the di- and tri-sialylated N-glycan content and total sialylated glycans content of the fusion protein composition obtained in Example I, Example II, and Example III.

TABLE 2
CTLA-4-IgG fusion protein composition at day 14

| | % Di and tri-sialylated | % Total sialylated | Titer |
|---|---|---|---|
| Example | N-glycans content | glycans | (g/L) |
| I | 30.74 ± 0.63 | 59.88 ± 0.07 | 5.33 ± 0.14 |
| II | 29.20 ± 0.78 | 60.27 ± 0.47 | 4.80 ± 0.14 |
| III | 20.83 ± 1.52 | 50.82 ± 2.01 | 4.58 ± 0.09 |

1. A cell culture method for the production of a fusion protein composition comprising sialylated N-glycans, the said method comprising
a) providing/culturing mammalian cells expressing the said fusion protein,
b) culturing the cells at a stringent pH value,
c) maintaining higher pCO₂ levels during the production phase than the pCO₂ levels during the growth phase
d) culturing the cells at first temperature for a first period of time, subjecting the cell culture to a temperature shift, wherein the second temperature is lower than the first temperature
e) supplementing the cell culture medium with at least one sugar
f) recovering the said fusion protein composition from the culture,
wherein, the sialylated N-glycan content of the fusion protein composition is reduced as compared to sialylated N-glycan content of the fusion protein composition produced by a similar method devoid of step b), step c) and step d).

2. The cell culture method as claimed in claim 1, wherein the reduced in sialylated N-glycan content is about 21% to 38%.

3. The cell culture method as claimed in claim 1, wherein stringent pH value is pH of 7.0±0.1.

4. The cell culture method as claimed in claim 1, wherein the pCO₂ levels as claimed in claim 1 is maintained at about 20 mmHg to about 50 mmHg during the growth phase and increased to 100 to 135 mmHg during the production phase.

5. The cell culture method as claimed in claim 1, wherein the temperature shift is from about 37° C. to 34° C. on day 6 of cell culture.

6. A cell culture method for the production of a fusion protein composition comprising sialylated N-glycans, the said method comprising
a) providing/culturing mammalian cells expressing the said fusion protein,
b) culturing the cells at a pH of 7.0±0.1,
c) maintaining the growth phase pCO₂ levels at about 20 mmHg to about 50 mmHg and increasing the pCO₂ levels to 100 to 135 mmHg during the production phase
d) subjecting the cell culture to a temperature shift from about 37° C. to 34° C. on day 6 of cell culture
e) supplementing the cell culture medium with at least one sugar
f) recovering the said fusion protein composition from the culture, whereby the resultant fusion protein composition comprises about 20.83% di- and tri-sialylated N-glycans content and/or about 50.82% total sialylated glycans.

7. The cell culture method as claimed in claim 1, wherein the fusion protein is a CTLA-4-IgG fusion protein.

8. The cell culture method as claimed in claim 1, wherein the fusion protein is abatacept.

9. The cell culture method as claimed in claim 1, wherein the mammalian cells used to express the fusion protein is CHO cells.

10. The cell culture method as claimed in claim 6, wherein the fusion protein is a CTLA-4-IgG fusion protein.

11. The cell culture method as claimed in claim 6, wherein the fusion protein is abatacept.

12. The cell culture method as claimed in claim 6, wherein the mammalian cells used to express the fusion protein is CHO cells.
